(19) United States
(12) Patent Application Publication
(10) Pub. No.: US 2004/0235900 A1
(43) Pub. Date: Nov. 25, 2004

(54) Antimicrobial oxazolifinones with improved pharmacokinetic profile and safety advantages

(76) Inventors: Yati Chugh, Chikalthana Aurangabad (IN); Nitin Shetty, Chikalthana Aurangabad (IN); Prasad K Deshpande, Chikalthana Aurangabad (IN); Milind D Sindkhedkar, Chikalthana Aurangabad (IN); Mohammad A Jafri, Chikalthana Aurangabad (IN); Ravinda D Yeole, Chikalthana Aurangabad (IN); Milind C Shukla, Chikalthana Aurangabad (IN); Shirikant V Gupte, Chikalthana Aurangabad (IN); Mahesh V Patel, Chikalthana Aurangabad (IN); Noel J De Souza, Chikalthana Aurangabad (IN)

Correspondence Address: LADAS & PARRY, 26 WEST 61ST STREET, NEW YORK, NY, 10023, US

(21) Appl. No.: 10/475,735

(22) PCT Filed: Jul. 10, 2003
(86) PCT No.: PCT/IN03/00237

(30) Foreign Application Priority Data
Jul. 11, 2002 (US) 60/395164
Apr. 21, 2003 (IN) 392/MUM/2003

(51) Int. Cl. C07D 413/02; A61K 31/454

(52) U.S. Cl. 514/326; 546/209

(57) ABSTRACT

The present invention provides agents having high antimicrobial activity for preventing and treating infectious diseases. Thus, the present invention provides novel cyanoalkylpiperidinophenyl oxazolidinone derivatives having antimicrobial activity, favourable pharmacokinetic and safety profiles, processes for making the compounds, as well as antimicrobial compositions containing said derivatives as active ingredients and methods of treating microbial infections with the said derivatives.

### FIELD OF THE INVENTION

The present invention relates to the field of novel cyanoalkylpiperidinophenyl oxazolidinones having antibacterial activity and favourable pharmacokinetic and safety profiles. The invention also relates to processes for making the compounds, to pharmaceutical compositions containing the compounds and to methods of treating bacterial infections with the compounds.

### BACKGROUND OF THE INVENTION

Oxazolidinones represent a chemical class of synthetic antimicrobial agents. Following a checkered historical development since about the early-1980s, a watershed event took place with the clinical development and release for medical use in the late 2000s of the first representative, Linezolid, of this class^{1,2} The unique properties of the members of this class of oxazolidinones is that they display activity against important Gram-positive human and veterinary pathogens including methicillin-resistant Staphylococcus aureus (MRSA), vancomycin resistant enterococci (VRE) and β-lactam resistant Streptococcus pneumoniae (PRSP). The oxazolidinones also show activity against Gram-negative aerobic bacteria and Gram-positive and Gram-negative anaerobes³.

The deficiencies of this class of oxazolidinones are that (a) they are inactive against Enterobacteriaceae⁴; (b) they are generally bacteriostatic and do not display activity at a useful level against aerobic fastidious Gram-negative pathogens, as well as Gram-negative anaerobes; (c) their borderline potency range for atypical respiratory pathogens such as Mycoplasma pneumoniae, M. hominis, Ureaplasma urealyticium and Chlamydia species limits their utility for respiratory tract infections³.

Our pending U.S. application Ser. No. 60/395,164 discloses a novel series of oxazolidinones which display increased potency, and incorporate bactericidal activity, in contrast to the earlier-described bacteriostatic activity of Linezolid and literature described oxazolidinones. Unusual bactericidal activity is shown to be displayed not just against Linezolid-sensitive strains but also for the first time against Linezolid-resistant strains, thus indicating a differential binding at conventional site/s of the ribonucleoprotein and/or targeting multiple such receptor sites.

PCT Publication Number WO 95/25106, corresponding U.S. Pat. No. 5,668,286 and related family Patent EP 0750 618 B1 disclose phenyl oxazolidinones and, in particular, substituted piperidinophenyl oxazolidinones, and their usefulness as antimicrobial agents. For the compounds of the invention, the MIC (minimum inhibitory concentration) data has been reported. Also for four compounds oral ED₅₀ values have been reported. None of the compounds in the instant invention have been actually previously prepared or described in U.S. Pat. No. 5,668,286 and EP 0750 618 B1. Also no data has been previously provided on their pharmacokinetic profile or on their safety profiles, in particular on their lack of myelosuppression forming potential.

It is important that for an antibacterial to be useful clinically, and thus to have industrial utility, it should have a favourable pharmacokinetic (PK) profile. Such a profile is governed by the levels of antibacterial circulating in the blood of the human or animal in which it is administered. Parameters usually in use to characterise the PK profile are the concentration (Cmax value) in the blood stream, its half life (T1 /2), and its area under the blood serum concentration curve (AUC). Additional parameters are the volume of distribution and clearance. From an analysis of these parameters, estimates are usually made of the patterns of mammalian/human dosage regimen. Drugs which have potential to be once-a-day, should have long serum half life and presence of circulating drug at therapeutic levels at extended time points such as 8 hrs, 10 hrs and 12 hrs. following a single administration, thereby ensuring that at least 50% of the time, i.e. 12 hrs. in case of a once-a-day drug, the drug remains at concentration higher than the minimal inhibition concentration against the pathogenic bacteria. A once-daily dosage regimen is more conducive to patient compliance with consequences of improved therapeutic benefits. Other factors in addition to a good PK profile are also of importance, including in particular the need for the oxazolidinone compound to be more safe or less toxic, especially in regard to its lower propensity to induce myelosuppression which results in toxic consequences of anemia, leucopenia, pancytopenia and trombocytopenia.

Hitherto, the only oxazolidinone in current clinical use is recommended for clinical usage to be administered twice-a-day (cf. package insert in marketed product named Zyvox (Linezolid). A caution also stated in the package itself and generally described in the literature for the class of oxazolidinone antibiotics is the propensity of the marketed drug and of other members of the class to induce myelosuppression.

There is a distinct need for an oxazolidinone which while being antibacterially efficatious, is capable of being administered as a once-a-day dosage regimen and have the safety advantages of being less myelosuppressive than currently available oxazolidinones in clinical use.

The present inventors have found that the novel cyanoalkylpiperidinophenyl oxazolidinones of the invention herein described have a pharmacokinetic profile and safety advantages which permit their suitability for a once-a-day dosage regimen and a more safe profile.

The following publications may be referred to with respect to the statements made in the above-described background information.

¹ Slee A M, et al., Antimicrob. Agents Chemother (1987) 31:1791 -1797;

² 2^{nd} European Congress of Chemotherapy and 7^{th} Biennial Conference on Antiinfective Agents and Chemotherapy (Final Program), (1998): 93;

³ Diekema D J et al., Lancet 2001; 358: 1975-82;

⁴ Zhanel G G et al., Canadian Journal of Infectious Diseases, 2001, 12: 379-390.

### SUMMARY OF THE INVENTION

The object of the present invention is to provide novel cyanoalkylpiperidinophenyl oxazolidinones or pharmaceutically acceptable salts or complexes thereof, which besides having high antimicrobial activity, have newer favourable pharmacokinetic profiles viz. Cmax, T½ and AUC values, and safety advantages, viz. lower propensity to induce myelosuppression.

The present inventors conducted intensive studies in order to accomplish the above object. As a result useful and novel oxazolidinone derivatives are found and the present invention has been accomplished on the basis of the findings.

The present invention provides novel cyanoalkylpiperidinophenyl oxazolidinones represented by the general Formula-I

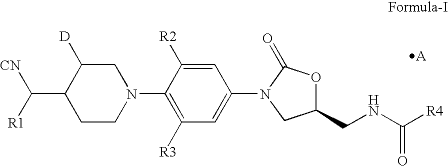

wherein,

R₁ is —H, C₁-C₈ alkyl, substituted alkyl, —COOH, —CN;

R₂ and R₃ are the same or different and are H or fluorine;

R₄ is H, C₁-C₈ alkyl, substituted C₁-C₈ alkyl, C₁-C₈ alkoxy,

D is H; C₁-C₈ alkyl, fluorine.

A is nothing, complex forming agent, organic base, amino acid.

The present invention also provides an antimicrobial agent that contains the oxazolidinone derivative or a pharmacetuically acceptable salt thereof as an effective ingredient. The antimicrobial agent containing the effective ingredient of the present invention can be used for treatment or prevention of infectious diseases. The term “treatment” as used herein means partial or total avoidance of symptoms of a disease in a patient who, according to a doctor's diagnosis, may suffer from the disease or a related state unless the preventive measure is taken. The compounds of this invention may be used to prevent infectious diseases by administering the compound to a human or animal that is at a risk for developing an infectious disease such as a health care worker, surgical patient, etc.

This invention provides novel oxazolidinone derivatives useful as preventatives and therapeutics for infectious diseases. The compounds of this invention have excellent antimicrobial action against various human and veterinary pathogens, including multiply-resistant staphylococci and streptococci, as well as anaerobic organisms such as bacteroides and clostridia species, and acid-fast Mycobacterium tuberculosis and M. avium. In particular, a special embodiment of the invention is that the compounds of the invention have a pharmacokinetic profile which provides a hitherto-unavailable once-a-day treatment potential for this class of oxazolidinone antiinfective agent. Another embodiment of the invention is that the compounds of the invention provides greater safety in respect of myelosuppression, known to be a class-specific hazard for this class of oxazolidinone antiinfective agent. The compounds can be used to prevent and or treat systemic or topical bacterial infections.

### DETAILED DESCRIPTION OF TERMS

“C1-C8 alkyl” means carbon atom chains having C1-C8 number of carbon atoms such as methyl, ethyl, propyl, butyl, pentyl, hexyl, heptyl, octyl. “Substituted alkyl” means C1-C8 alkyl, bearing substituents like one or more hydroxy, methane sulfonyloxy or halogen atoms such as fluorine, chlorine, bromine.

C1-C8 alkyloxy stands for methoxy, ethoxy, propoxy, butoxy, pentoxy, hexyloxy, heptyloxy, octyloxy and isomeric forms thereof.

Complex forming agents stands for agents which can form complex with oxazolidinones such as cyclodextrins.

Cyclodextrin can be selected from α-cyclodextrin, β-cyclodextrin, γ-cyclodextrin.

β-Cyclodextrin can be further selected from methyl-β-cyclodextrin, 2-hydroxy-propyl-β-cyclodextrin (2-HP-β-cyclodextrin), 3-hydroxy-propyl-β-cyclodextrin (3-HP-β-cyclodextrin), sulfobutylether-β-cyclodextrin.

Organic bases stands for bases such as ethanolamine, guanidine etc. Amino acid stands for dibasic amino acids such as racemic or optically active arginine, and lysine.

The preferred absolute configuration at C-5 of the oxazolidinone ring of compounds claimed in this invention is as represented in the structure of Formula I. This absolute configuration is called (S) under the Cahn-Ingold-Prelog nomenclature system. It is this (S)-enantiomer which is pharmacologically active. The racemic mixture is useful in the same way and for the same purpose as the pure (S)-enantiomer; the difference is that twice as much racemic material must be used to produce the same antibacterial effect. Depending on substituents, the compounds of this invention may exist in geometric, optical and other isomeric forms and this invention embraces any of these isomers.

Particular preferred examples of the oxazolidinone derivatives represented by the general Formula I are as in the following list:

1. (S)-N-{3-[4-(4-cyanomethyl-piperidin-1-yl)-phenyl]-2-oxo-oxazolidin-5-ylmethyl}-formamide;

2. (S)-N-{3-[4-(4-cyanomethyl-piperidin-1-yl)-phenyl]-2-oxo-oxazolidin-5-ylmethyl}-acetamide;

3. (S)-N-{3-[4-(4-cyanomethyl-3-fluoropiperidin-1-yl)-phenyl]-2-oxo-oxazolidin-5-ylmethyl}-acetamide;

4. (S)-N-{3-[4-(4-cyanomethyl-piperidin-1-yl)-3-fluoro-phenyl]-2-oxo-oxazolidin-5-ylmethyl}-acetamide;

5. (S)-N-{3-[4-(4-cyanomethyl-piperidin-1-yl)-3-fluoro-phenyl]-2-oxo-oxazolidin-5-ylmethyl}-acetamide, inclusion complex with 3-hydroxy-propyl-β-cyclodextrin.

6. (S)-N-{3-[4-(4-cyanomethyl-piperidin-1-yl)-3-fluoro-phenyl]-2-oxo-oxazolidin-5-ylmethyl}-propionamide;

7. (S)-N-{3-[4-(4-cyanomethyl-piperidin-1-yl)-3-fluoro-phenyl]-2-oxo-oxazolidin-5-ylmethyl}-dichloroacetamide;

8. (S)-N-{3-[4-(4-cyanomethyl-piperidin-1-yl)-3-fluoro-phenyl]-2-oxo-oxazolidin-5-ylmethyl}-trifluoroacetamide;

9. (S)-N-{3-[4-(4-cyanomethyl-piperidin-1-yl)-3-fluoro-phenyl]-2-oxo-oxazolidin-5-ylmethyl}-ethylcarbamate;

10. (S)-N-{3-[4-(4-cyanomethyl-piperidin-1-yl)-3-fluoro-phenyl]-2-oxo-oxazolidin-5-ylmethyl}-iso-butylcarbamate;

11. (S)-N-{3-[4-(4-cyanomethyl-piperidin-1-yl)-3-fluoro-phenyl]-2-oxo-oxazolidin-5-ylmethyl}-t-butylcarbamate;

12. (S)-N-{3-[4-((4-cyanomethyl)-3-methyl-piperidin-1-yl)-3-fluoro-phenyl]-2-oxo-oxazolidin-5-ylmethyl}-acetamide;

13. (S)-N-{3-[4-((4-cyanomethyl)-3-fluoro-piperidin-1-yl)-3-fluoro-phenyl]-2-oxo-oxazolidin-5-ylmethyl}-isobutylcarbamate;

14. (S)-N-{3-[4-(4-(1-cyanoethyl)-piperidin-1-yl)-3-fluoro-phenyl]-2-oxo-oxazolidin-5-ylmethyl}-acetamide;

15. (S)-N-{3-[4-(4-(1-cyanopropyl)-piperidin-1-yl)-3-fluoro-phenyl]-2-oxo-oxazolidin-5-ylmethyl}-acetamide;

16. (S)-N-{3-[4-(4-(1-cyanobutyl)-piperidin-1-yl)-3-fluoro-phenyl]-2-oxo-oxazolidin-5-ylmethyl}-acetamide;

17. (S)-N-{3-[4-(4-(1-cyano-2-hydroxyethyl)-piperidin-1-yl)-3-fluoro-phenyl]-2-oxo-oxazolidin-5-ylmethyl}-acetamide;

18. (S)-N-{3-[4-(4-(1-cyano-1-hydroxycarbonyl)-piperidin-1-yl)-3-fluoro-phenyl]-2-oxo-oxazolidin-5-ylmethyl}-acetamide;

19. (S)-N-{3-[4-(4-(1,1-dicyanomethyl)-piperidin-1-yl)-3-fluoro-phenyl]-2-oxo-oxazolidin-5-ylmethyl}-acetamide;

The compounds represented by the general Formula I can be prepared by the method of reaction Scheme 1.

All the starting materials are prepared by procedures described in this Scheme-1 or by procedures that would be well known to one of ordinary skill in organic chemistry. The variables used in Scheme-1 are as defined above. Optically pure material could be obtained by one of a number of asymmetric synthesis or alternatively by resolution from a racemic mixture.

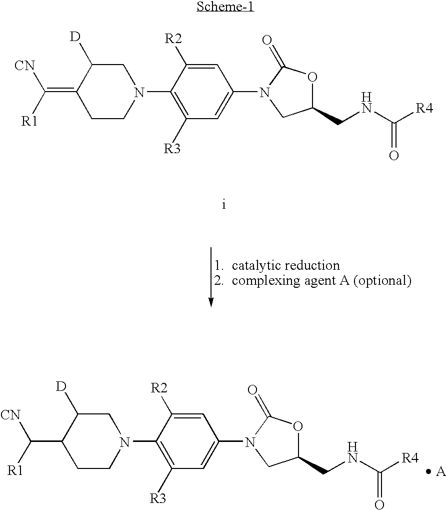

In accordance with Scheme-1, cyanoalkylidene oxazolidinone intermediate i (prepared as described in our U.S. Provisional Application 60/395,164 which in-turn prepared from an intermediate synthesised as per procedure described in U.S. Pat. No. 5,668,286) upon reduction in the presence of a catalyst such as 5% palladium on carbon, 10% palladium on carbon, palladium hydroxide at atmospheric pressure of hydrogen gas or alternatively in the presence of hydrogen sources such as ammonium formate, cyclohexene in a suitable solvent such as ethyl acetate, tetrahydrofuran, methanol, or mixture thereof at a temperature between 20° C. to 50° C. provides the cyanoalkyl compound of the Formula I of the invention. This compound was optionally treated with a suitable complex forming agent such as α-cyclodextrin, β-cyclodextrin, γ-cyclodextrin or guanidine in a suitable solvent such as water, methanol, acetone and mixture thereof to provide a cyclodextrin complex of a compound of Formula I of the invention.

### General Methods

### General Method to Prepare Oxazolidinone

A. Compound of Formula I is Prepared By

i) stirring the cyanoalkylidene compound i in the presence of catalyst such as 5% palladium on carbon, 10% palladium on carbon, palladium hydroxide preferably 10% palladium on carbon; at atmospheric pressure of hydrogen gas or alternatively in the presence of a hydrogen source such as ammonium formate, cyclohexene preferably in the presence of hydrogen gas; in a suitable solvent such as ethyl acetate, tetrahydrofuran, methanol, or mixture thereof preferably tetrahydrofuran at a temperature between 20° C. to 50° C. to provide the cyanoalkyl compound of the Formula I of the invention.

ii) Optionally stirring the cyanoalkyl compound obtained in step i with a suitable complex forming agent such as α-cyclodextrin, β-cyclodextrin, substituted β-cyclodextrin, γ-cyclodextrin preferably with substituted β-cyclodextrin; in a suitable solvent such as water, methanol, acetone and mixture thereof preferably water; at a temperature between 30° C. to 60° C. for 2 to 48 hours preferably 24 hours followed by evaporating the solvent under reduced pressure and drying the compound under vacuum to provide a cyclodextrin complex of the compound of Formula I of the invention.

### Method 1

### Preparation of 1-cyano Substituted Alkyl Oxazolidinones of the Invention

### Preparation of (S)-N-{3-[4-(4-(1-cyano substitued/unsubstituted alkyl)-piperidin-1-yl)-3-fluoro-phenyl]-2-oxo-oxazolidin-5-ylmethyl}-acetamide/-alkylcarbamate

A clear solution made of the cyanoalkylidene oxazolidinone in tetrahydrofuran and 10% palladium on carbon was stirred under atmospheric hydrogen pressure at a temperature between 20 to 50° C.

After completion of the reaction the catalyst was filtered and the filtrate was concentrated to dryness under vacuum. The product thus obtained was chromaographed on silica gel to provide the 1-cyano substituted alkyl oxazolidinone of the invention.

### Method 2

### Preparation of 3-hydroxy-propyl-β-cyclodextrin Inclusion Complex With Oxazolidinone of the Invention in a Molar Ratio 1:1.12 or 1:2.0

To a clear solution of 3-HP-β-CD (0.112 mmol or 0.2 mmol) in 10 to 15 ml of distilled water, was charged 1-cyano substituted alkyl oxazolidinone (0.1 mmol) at a temperature between 20 to 50° C. under stirring. The suspension was stirred at 40 to 60° C. temperature for 0.5 to 4 hours to obtain a clear solution. The clear solution was allowed stand at a temperature between 20 to 40° C. for 12 to 24 hours. The reaction mixture was filtered and filtrate was evaporated under vacuum at a temperature below 60° C. to provide a compound of the invention, typically in 80 to 98% yield.

The compounds of the invention are useful for the treatment of microbial infections in humans and other warm blooded animals by parenteral, oral, topical administration or by other means of administration.

The present invention encompasses certain compounds, dosage forms, and methods of administering the compounds to a human or other animal subject. Specific compounds and compositions to be used in the invention must, accordingly, be pharmaceutically acceptable. As used herein, such a “pharmaceutically acceptable” component is one that is suitable for use with humans and/or animals without undue adverse side effects (such as toxicity, irritation, and allergic response) commensurate with a reasonable benefit/risk ratio.

The pharmaceutical compositions are prepared according to conventional procedures used by persons skilled in the art to make stable and effective compositions. In the solid, liquid, parenteral and topical dosage forms, an effective amount of the active compound or the active ingredient is any amount, which produces the desired results.

For the purpose of this invention the pharmaceutical compositions may contain the active compounds of the invention, their derivatives, salts and hydrates thereof, in a form to be administered alone, but generally in a form to be administered in admixture with a pharmaceutical carrier selected with regard to the intended route of administration and standard pharmaceutical practice. Suitable carriers which can be used are, for example, diluents or excipients such as fillers, extenders, binders, emollients, wetting agents, disintegrants, surface active agents and lubricants which are usually employed to prepare such drugs depending on the type of dosage form.

Any suitable route of administration may be employed for providing the patient with an effective dosage of the compound of the invention their derivatives, salts and hydrates thereof. For example, oral, rectal, vaginal, parenteral (subcutaneous, intramuscular, intravenous), nasal, transdermal, topical and like forms of administration may be employed. Dosage forms include (solutions, suspensions, etc) tablets, pills, powders, troches, dispersions, suspensions, emulsions, solutions, capsules, injectable preparations, patches, ointments, creams, lotions, shampoos and the like.

The prophylactic or therapeutic dose of the compounds of the invention, their derivatives, salts or hydrates thereof, in the acute or chronic management of disease will vary with the severity of condition to be treated, and the route of administration. In addition, the dose, and perhaps the dose frequency, will also vary according to the age, body weight and response of the individual patient. In general, the total daily dose range, for the compounds of the invention, the derivatives, salts or hydrates thereof, for the conditions described herein, is from about 200 mg to about 1500 mg, in single or divided doses. Preferably, a daily dose range should be between about 400 mg to 1200 mg, in single or divided dosage, while most preferably a daily dose range should be between about 500 mg to about 1000 mg in divided dosage. While intramuscular administration may be a single dose or up to 3 divided doses, intravenous administration can include a continuous drip. It may be necessary to use dosages outside these ranges in some cases as will be apparent to those skilled in the art. Further, it is noted that the clinician or treating physician will know how and when to interrupt, adjust, or terminate therapy in conjunction with individual patient's response. The term “an amount sufficient to eradicate such infections but insufficient to cause undue side effects” is encompassed by the above-described dosage amount and dose frequency schedule. “Antibacterially effective amount” is the amount required to provide a desirable biological effect of restricting the growth of bacteria or killing bacteria.

A specific embodiment of this invention is that the pharmacokinetic profile of a compound of the invention is such that it permits administration of a dosage schedule which is a much-desired once-a-day dosing, a schedule not so far advocated for the only currently available drug in the market. A further embodiment of this invention is that the once-a-day dosage schedule confirs safety advantages in respect of the phenomenon of myelosuppression described as an attribute of these class of compounds which needs to be avoided.

Pharmaceutical compositions of the present invention suitable for oral administration may be presented as discrete units such as capsules, cachets, or tablets, or aerosol sprays, each containing a predetermined amount of the active ingredient, as a powder or granules, or as a solution or a suspension in an aqueous liquid, a non-aqueous liquid, an oil-in-water emulsion, or a water-in-oil liquid emulsion. Such compositions may be prepared by any of the methods of pharmacy, but all methods include the step of bringing into association the active ingredient with the carrier, which constitutes one or more necessary ingredients. In general, the compositions are prepared by uniformly and intimately admixing the active ingredient with liquid carriers or finely divided solid carriers or both, and then, if necessary, shaping the product into the desired presentation.

The compositions of the present invention include compositions such as suspensions, solutions, elixirs, aerosols, and solid dosage forms. Carriers as described in general above are commonly used in the case of oral solid preparations (such as powders, capsules and tablets), with the oral solid preparations being preferred over the oral liquid preparations. The most preferred oral solid preparation is tablets.

Because of their ease of administration, tablets and capsules represent the most advantageous oral dosage unit form, in which case solid pharmaceutical carriers are employed. Examples of suitable carriers include excipients such as lactose, white sugar, sodium chloride, glucose solution, urea, starch, calcium carbonate, kaolin, crystalline cellulose and silicic acid, binders such as water, ethanol, propanol, simple syrup, glucose, starch solution, gelatin solution, carboxymethyl cellulose, shellac, methyl cellulose, potassium phosphate and polyvinyl pyrrolidone, disintegrants such as dried starch, sodium alginate, agar powder, laminaria powder, sodium hydrogen carbonate, calcium carbonate, Tween (fatty acid ester of polyoxyethylenesorbitan), sodium lauryl sulfate, stearic acid monoglyceride, starch, and lactose, disintegration inhibitors such as white sugar, stearic acid glyceryl ester, cacao butter and hydrogenated oils, absorption promoters such as quaternary ammonium bases and sodium lauryl sulfate, humectants such as glycerol and starch, absorbents such as starch, lactose, kaolin, bentonite and colloidal silicic acid, and lubricants such as purified talc, stearic acid salts, boric acid powder, polyethylene glycol and solid polyethylene glycol.

The tablet, if desired, can be coated, and made into sugar-coated tablets, gelatin-coated tablets, enteric-coated tablets, film-coated tablets, or tablets comprising two or more layers.

If desired, tablets may be coated by standard aqueous or non-aqueous techniques.

In molding the pharmaceutical composition into pills, a wide variety of conventional carriers known in the art can be used. Examples of suitable carriers are excipients such as glucose, lactose, starch, cacao butter, hardened vegetable oils, kaolin and talc, binders such as gum arabic powder, tragacanth powder, gelatin, and ethanol, and disintegrants such as laminaria and agar.

In molding the pharmaceutical composition into a suppository form, a wide variety of carriers known in the art can be used. Examples of suitable carriers include polyethylene glycol, cacao butter, higher alcohols, gelatin, and semi-synthetic glycerides.

A second preferred method is parenterally for intramuscular, intravenous or subcutaneous administration.

A third preferred route of administration is topically, for which creams, ointments, shampoos, lotions, dusting powders and the like are well suited. Generally, an effective amount of the compound according to this invention in a topical form is from about 0.1% w/w to about 10% w/w of the total composition. Preferably, the effective amount of the compound of the invention is 1% w/w of the total composition.

In addition to the common dosage forms set out above, the compounds of the present invention may also be administered by controlled release means and/or delivery devices such as those described in U.S. Pat. Nos. 3,845,770; 3,916,899; 3,536,809; 3,598,123 and 4,008,719; the disclosures of which are hereby incorporated by reference.

Desirably, each tablet contains from about 200 mg to about 1500 mg of the active ingredient. Most preferably, the tablet, cachet or capsule contains either one of three dosages, about 200 mg, about 400 mg, or about 600 mg of the active ingredient.

When the pharmaceutical composition is formulated into an injectable preparation, in formulating the pharmaceutical composition into the form of a solution or suspension, all diluents customarily used in the art can be used. Examples of suitable diluents are water, ethyl alcohol, polypropylene glycol, ethoxylated isostearyl alcohol, polyoxyethylene sorbitol, and sorbitan esters. Sodium chloride, glucose or glycerol may be incorporated into a therapeutic agent.

The antimicrobial pharmaceutical composition may further contain ordinary dissolving aids, buffers, pain-alleviating agents, and preservatives, and optionally coloring agents, perfumes, flavors, sweeteners, and other drugs.

For topical application, there are employed as non-sprayable forms, viscous to semi-solid or solid forms comprising a carrier compatible with topical application and having a dynamic viscosity preferably greater than water. Suitable formulations include but are not limited to solutions, suspensions, emulsions, creams, ointments, powders, liniments, salves, aerosols, etc., which are, if desired, sterilized or mixed with auxiliary agents, e.g. preservatives, antioxidants, stabilizers, wetting agents, buffers or salts for influencing osmotic pressure, etc. For topical application, also suitable are sprayable aerosol preparations wherein the active ingredient preferably in combination with a solid or liquid inert carrier material.

A specific embodiment of the invention is the preparation of storage stable compositions of the compounds of the invention of formula I. Such stable compositions can be advantageously made through the use of selective stabilizers. Different stabilizers are known to those skilled in the art of making pharmaceutical compositions. Of special utility for making storage stable compositions of the compound of the invention of formula I, stabilizers such as disodium ethylenediaminetetraacetic acid (EDTA), tromethamine, cyclodextrins such as gamma-cyclodextrin, hydroxy-propyl-gamma-cyclodextrin have been found to be useful.

In a specific embodiment of the invention, the pharmaceutical compositions contain an effective amount of the active compounds of the invention, its derivatives, inclusion complexes, salts or hydrates thereof described in this specification as hereinbefore described in admixture with a pharmaceutically acceptable carrier, diluent or excipients, and optionally other therapeutic ingredients.

The invention is further defined by reference to the following examples describing in detail the preparation of the composition of the present invention as well as their utility. It will be apparent to those skilled in the art that many modifications, both to materials and methods may be practiced without departing from the purpose and scope of this invention.

The compounds of this invention are useful antimicrobial agents, effective against various human and veterinary pathogens, similar to the efficacy described for the compounds of PCT WO 95/25106, U.S. Pat. No. 5,668,286 and EP 0 750, 618 B1.

The test methods used for verifying the antimicrobial action of compound within the scope of this invention are essentially the same as those described in PCT WO 95/25106, U.S. Pat. No. 5,668,286 and EP 0 750, 618 B1, with the difference that the strains of the organisms used for the MIC determinations in these patents and applications are Staphylococcus aureus ATCC 25923, Staphylococcus epidermidis ATCC 12828, Streptococcus pyogenes ATCC 19615.

### Test Example 1

The in-vitro MIC methods of test compounds were determined essentially as described in PCT WO 95/25106, U.S. Pat. No. 5,668,286 and EP 0 750 618 B1.

### MIC Test Method

Overnight grown cultures of S. aureus organisms in Tryptic Soya broth were diluted in Mueller Hinton Broth to give optical density matching with MacFarland tube 0.5 standard. Cultures were further diluted 1:10 in Mueller Hinton broth. Using Denley's mutipoint inoculator, 10⁴ cells were deposited on Mueller Hinton agar (Difco) containing range of 2 fold dilutions of test compounds. These plates were incubated for 24 hrs at 35° C. and MIC results recorded. MIC is defined as minimum drug concentration that inhibits test organisms. For determining MIC of test compounds against Streptococcus pneumoniae, Mueller Hinton agar containing 5% sheep blood was employed.

Results:

TABLE 1
Minimum Inhibitory Concentrations (MIC, μg/ml)

| Compound No. | MRSA-32 | E. faecalis | S. pneumoniae |
|---|---|---|---|
| 2 | 2.0 | 2.0 | 2.0 |
| 4 | 2.0 | 2.0 | 1.0 |
| 8 | 4.0 | 8.0 | 2.0 |
| 10 | >16.0 | ND | ND |
| 12 | 2.0 | 2.0 | 2.0 |
| 14 | 2.0 | 2.0 | 2.0 |
| 15 | 4.0 | 4.0 | 2.0 |
| 16 | 4.0 | 4.0 | 4.0 |
| 17 | 2.0 | 2.0 | 2.0 |
| 19 | 2.0 | 2.0 | 1.0 |
| Linezolid | 2.0 | 2.0 | 0.5 |

Methicillin Resistant Staphylococcus aureus MRSA-32,
Enterococcus faecalis ATCC 29212
Streptococcus pneumoniae ATCC 49619

The results obtained for the compounds of the invention show that they have potent antibacterial activity.

The antimicrobial action of the compounds of this invention was also verified by the Murine Assay procedure (in vivo) as described in PCT WO 95/25106, U.S. Pat. No. 5,668,286 and EP 0 750, 618 B1 for the compounds cited in the aforementioned patents as well as for the instant compounds of the invention.

### Test Example 2

### Murine Assay Procedure

Oxazolidinone new chemical entities (NCEs) were evaluated for their in vivo efficacy in a murine infection caused by multi-drug resistant, methicillin resistant Staphylococcus aureus strain, referred to as MRSA 32, a clinical isolate obtained from a hospitalised patient. The procedure used for the murine assay is as follows:

Four weeks old swiss mice of 18-22 gm body weight were infected with MRSA 32 strain suspended in 5% Hog gastric Mucin. The infecting dose of bacteria was set at 1-2×10⁸ CFU/animal. The infecting dose was administered in 0.5 ml volume injected into peritonial cavity of mice. The treatment with oxazolidinone NCEs was started one hour after infection by administering 100-200 μl of the suspensions of oxazolidinone compounds in 5% Tween 80 by oral gavage. A repeat dose was similarly administered 3 hrs later. Each oxazolidinone NCE was tested at 2-3 different dosages in the range of 2.5 mg/kg to 20 mg/kg. In each dose group 6 mice were included. As an infection control group 12 mice were infected with MRSA 32 strain without giving any treatment. Normally untreated infected mice die within 24 hrs. due to the systemic spread of infection through out the animal body. Those oxazolidinone compounds which were orally bioavailable and share the attributes of good potency, balanced serum protein binding and metabolic stability demonstrate in vivo efficacy by protecting the MRSA 32 infected mice at therapeutically rationale doses in the range of 5-20 mg/kg. For compounds affording protection of MRSA infected mice, ED₅₀ dosages were calculated on the basis of percentage survival on Day 7 after infection.

The in-vivo ED₅₀ values using the test compounds were determined essentially by the method as described in PCT WO 95/25106, U.S. Pat. No. 5,668,286 and EP 0 750 618 B1.

Results:

Compounds with numbers 2, 4 and 17 had ED₅₀ values 20.0 mg/kg, 2.5-5.0 mg/kg and 20.0 mg/kg respectively upon oral administration; hence they were as effective as the control linezolid.

A specific embodiment of this invention is that the pharmacokinetic profile of a compound of the invention is such that it permits administration of a dosage schedule which is a much-desired once-a-day dosing, a schedule not so far advocated for the only currently available drug in the market.

Pharmacokinetic parameters of a representative compound No. 4 of the invention viz. (S)-N-{3-[4-(4-cyanomethyl-piperidin-1-yl)-3-fluoro-phenyl]-2-oxo-oxazolidin-5-ylmethyl}-acetamide, were determined and compared with those of compounds Nos. 30, 3, 7 and 11 described in U.S. Pat. No. 5,668,286. Compound No.30 is (S)-N-{3-[4-(1,4-dioxa-8-aza-spiro[4.5]dec-8-yl)-3-fluoro-phenyl]-2-oxo-oxazolidin-5-ylmethyl}-acetamide, Compound No.3 is (S)-N-{3-[3-fluoro-4-(4-hydroxy-piperidin-1-yl)-phenyl]-2-oxo-oxazolidin-5-ylmethyl}-acetamide, Compound No.7 is (S)-N-{3-[3-fluoro-4-(4-oxo-piperidin-1-yl)-phenyl]-2-oxo-oxazolidin-5-ylmethyl}-acetamide, and Compound No.11 is (S)-N-{3-[3-fluoro-4-(4-hydroxyimino-piperidin-1-yl)-phenyl]-2-oxo-oxazolidin-5-ylmethyl}-acetamide. The choice of these compounds from U.S. Pat. No. 5,668,286 was made because compounds 30, 7 and 11 are compounds for which the said patent discloses ED₅₀ data in the murine assay. Compound 3 was selected because it bears a hydroxy substituent on the piperidino ring and is shown to have potent antibacterial MIC activity.

We now describe the test method for displaying and verifying the pharmcokinetic profile of the compounds within the scope of this invention which would enable bioavailability of the drug in mammals in such amounts that the dosing can be reduced to once-a-day.

### Test Example 3

### Pharmacokinetic Studies

Oral (5 mg/kg) and intravenous (5 mg/kg bolus) pharmacokinetic studies were done in dog. Blood samples were collected at time points of 0, 0.08 (not for oral), 0.25, 0.50, 1.0, 1.5, 2.0, 3.0, 4.0, 5.0, 6.0, 8.0, 10.0, 12.0 and 24.0 hours. To facilitate i.v. dosing and collection of blood samples, the dogs were implanted with cannula in cephalic vein. Serum obtained from blood samples was used for HPLC-based analysis.

Serum samples were extracted by solid phase extraction technique using Water's OASIS HLB cartridges. An HPLC-Diode array detection system was used for analysis. Prepared samples were chromatographed on a YMC-AM reversed phase column (150×4.6 mm ID; 5 μm) using an isocratic mobile phase acetate buffer (50 mmol ammonium acetate pH 6.6) acetonitrile, 66:34% v/v (for a representative compound of the invention, 68:32% v/v for compound Nos. 30, 3, 7 and 11 of U.S. Pat. No. 5,668,286, and 75:25% v/v for linezolid) at a flow rate of 1 ml/min, measured at λₘₐₓ 254 nm. Independently prepared analytical standards and quality control samples were analyzed with each set of unknown samples. The calculated pharmacokinetic parameters are shown in Tables 2 & 3.

Result:

The pharmacokinetics of a representative compound of the invention following single oral dose administration of the compound in beagle dog is shown in Table 2.

The pharmacokinetics of a representative compound of the invention following single I.V. bolus dose administration of the compound in beagle dog is shown in Table 3.

TABLE 2
Comparative PK Parameters following Single Oral dose in Beagle Dog
(5 mg/kg, p.o. Compound No. 4 and linezolid administered in 5% Tween and the other compounds administered in cyclodextrin)

| | | Compound cited in | Compound cited in | Compound cited in | Compound cited in | |
|---|---|---|---|---|---|---|
| | | PCT WO | PCT WO | PCT WO | PCT WO | |
| | Compound | 95/25106 (No. 30) | 95/25106 (No. 3) | 95/25106 (No. 7) | 95/25106 (No. 11) | |
| | No. 4 of | U.S. Pat. No. 5,668,286 | U.S. Pat. No. 5,668,286 | U.S. Pat. No. 5,668,286 | U.S. Pat. No. 5,668,286 | |
| | the invention | (No. 30) and | (No. 3) and | (No. 7) and | (No. 11) and | |
| PK parameter | (n = 3) | EP 0750618 B1 (No 28) | EP 0750618 B1 (No 3) | EP 0750618 B1 (No 7) | EP 0750618 B1 (No 11) | LNZ |
| Cmax (μg/ml) | 5.66 ± 1.49 | 5.31 | 1.52 | 0.0 | 0.0 | 5.70 ± 0.69 |
| C-12 hr | 2.84 ± 0.17 | 1.84 | 0.0 | 0.0 | 0.0 | 0.0 |
| (μg/ml) | | | | | | |
| T1/2 (hr) | 10.79 ± 2.45 | 6.68 | 1.37 | 0.0 | 0.0 | 2.42 ± 0.17 |
| AUC (0-24 hr) | 71.56 ± 0.25 | 47.25 | 2.74 | 0.0 | 0.0 | 22.83 ± 2.17 |
| μg · hr/ml | | | | | | |

LNZ: reference marketed compound Linezolid

TABLE 3
Comparative PK Parameters following Single I.V. Bolus Dose in Beagle Dog
(15 mg/kg Compound No. 4 and linezolid administered in 5% Tween and the other compounds administered in cyclodextrin)

| | | Compound cited in | Compound cited in | Compound cited in | Compound cited in | |
|---|---|---|---|---|---|---|
| | | PCT WO | PCT WO | PCT WO | PCT WO | |
| | | 95/25106 (No. 30) | 95/25106 (No. 3) | 95/25106 (No. 7) | 95/25106 (No. 11) | |
| | Compound | U.S. Pat. No. 5,668,286 | U.S. Pat. No. 5,668,286 | U.S. Pat. No. 5,668,286 | U.S. Pat. No. 5,668,286 | |
| | No. 4 of | (No. 30) and | (No. 3) and | (No. 7) and | (No. 11) and | |
| PK parameter | the invention | EP 0750618 B1 (No 28) | EP 0750618 B1 (No 3) | EP 0750618 B1 (No 7) | EP 0750618 B1 (No 11) | LNZ (n = 1) |
| Cmax (μg/ml) | 8.28 | 7.64 | 6.19 | 0.0 | 4.15 | 7.16 |
| C-12 hr | 2.64 | 1.19 | 0.0 | 0.0 | 0.0 | 0.0 |
| (μg/ml) | | | | | | |
| T1/2 (hr) | 12.95 | 6.71 | 0.98 | 0.0 | 0.16 | 2.41 |
| AUC (0-24 hr) | 80.94 | 45.08 | 7.53 | 0.0 | 1.32 | 23.26 |
| μg · hr/ml | | | | | | |

LNZ: reference marketed compound Linezolid

The pharmacokinetic values show the superiority of the compound of the invention over the compounds disclosed in PCT WO 95/25106, U.S. Pat. No. 5,668,286 and EP 0 750 618 B1. The values are in support of a potential use of the compounds of the invention for once-a-day treatment.

A further embodiment of this invention is that the once-a-day dosage schedule confirrs safety advantages in respect of the phenomenon of myelosuppression described as an attribute of this class of compounds which needs to be avoided.

Myelosuppression potential of a representative compound No.4 of the invention viz. (S)-N-{3-[4-(4-cyanomethyl-piperidin-1-yl)-3-fluoro-phenyl]-2-oxo-oxazolidin-5-ylmethyl}-acetamide, was determined and compared with that of compound No. 30 described in U.S. Pat. No. 5,668,286. Compound No. 30 is (S)-N-{3-[4-(1,4-dioxa-8-aza-spiro[4.5]dec-8-yl)-3-fluoro-phenyl]-2-oxo-oxazolidin-5-ylmethyl}-acetamide. The choice of this compound from U.S. Pat. No. 5,668,286 was made because it is a compound with the lowest ED₅₀ value of those cited in the said US patent.

We also, furthermore, describe below the test methods to determine the potential myelosuppression activity of the compounds of the invention in rats. In Table 4 is provided the values of the parameters assessed to determine the myelosuppressive activity potential. The results indicate that compound 4 of the invention has no or lower myelosuppressive potential in comparison to compound No.30 of U.S. Pat. No. 5,668,286 and the reference compound linezolid.

The test method for verifying the myelosuppression potential is described below. The results are shown in Table 4.

### Test Example 4

### Myelosuppressive Potential

Method

A group of 6 Wistar rats (3 male and 3 female) was exposed to a representative compound of the invention viz. compound No. 4 of the invention, by oral route at a single dose of 50 mg/kg per day for 14 consecutive days. Compound No.30 of U.S. Pat. No. 5,668,286 was also subjected to the same protocol. Linezolid (LNZ) was used as a comparator drug and was administered to rats (by oral route at a dose of 2×25 mg/kg per day for 14 consecutive days. Vehicle treated controls were maintained using identical experimental conditions. The treated as well as control rats were sacrificed 24 hr after the last dose.

One parameter measured was the spleen to body weight ratio and thymus to body weight ratio in treated versus control animals.

The spleen and thymus were trimmed free of fat and other contiguous organs/tissues and were weighed in an analytical balance (Sartorious BP 210). The spleen to terminal body weight ratio and the thymus to terminal body weights ratio was calculated to provide the respective relative weights. The ratio value of the respective relative weight of a treated animal versus the relative weight of a control animal is provided in Table 4. The myelosuppressive potential of a compound is inversely proportional to the ratio value. For instance a ratio less than 0.75 indicates myelosuppressive potential. The results shown in Table 4 clearly indicate that the representative compound of the invention is devoid of immunosuppression potential in contrast to the reference compound Linezolid.

A second parameter measured was the change in reticulocyte count, for which the following method was used.

### Reticulocyte Counts

Blood Collection

Blood was collected on day 15 (24 hours after the last dose administration) from all the treated as well as control rats by retro-orbital sinus puncture using clean glass rat capillary tubes. The blood was collected in sterile clean and anticoagulated Eppendorf microtubes. EDTA was used as the anticoagulant (conc.: 2 mg/10 ml).

Staining Procedure

The staining solution of New Methylene Blue (NMB) was prepared in iso-osmotic phosphate buffer pH 7.4 (150 mM) saline to achieve a concentration of 0.6% (w/v) and the stock stored in an amber colored glass bottle at 2-6° C.

Counting Procedure

3 slides/animal were prepared according to NCCLS staining procedure for reticulocyte staining and counting. The collected blood was mixed gently by inverting the tube 2-3 times and freshly prepared stock of 0.6% (w/v) NMB was mixed with the blood at equal volume in microtube and incubated at 37° C. for 20 minutes. The stained blood specimen was smeared evenly on a clean, dry and grease-free slide with the help of a spreader. 3 slides per rat were prepared, allowed to dry in warm air and mounted with the help of DPX solution and a clean cover slip. Counting of erythrocytes and reticulocyte was done for each slide using a microscope under 100× magnification (oil immersion). The percentage presence of reticulocyte was determined in 1000 erythrocytes and was expressed in terms of percentage of reticulocytes over erythrocytes. The ratio of the percentage reticulocytes in treated animals versus controlled animals is provided in Table 4 as a ratio value of the percentages. The myelosuppressive potential is inversely proportional to the ratio value. For instance a ratio less than 0.75 indicates myelosuppressive potential. The results provided in Table 4 clearly indicate that the compound No.4 of the invention is devoid of immunosuppressive potential. (Reference: The National Committee for Clinical Laboratory Standards (NCCLS): Methods for Reticulocyte Counting (Flow Cytometry and Supravital Dyes); Approved Guideline. NCCLS Document H44-A (ISBN 1-56238-302-7). NCCLS 940 West Vally Road, Suit 1400, Wayne, Pa. 19087-1898, USA, 1997. Page No. 04)

TABLE 4
Ratio value of spleen weight/body weight and thymus
weight/body weight of treated animal versus control animal,
and ratio of percentage reticulocytes of treated animal
versus control animal

| | | | Ratio of % |
|---|---|---|---|
| | Relative Weights Ratio | | reticulocytes |
| Compounds | Spleen | Thymus | Retics |
| Compound No. 4 of this | 0.99 | 0.99 | 1.00 |
| invention | (±0.0) | (±0.0) | (±0.00) |
| Compound No. 30 of | 0.94 | 0.97 | 0.59 |
| U.S. Pat. No. 5,668,258 | (±0.03) | (±0.04) | (±0.001) |
| LNZ | 0.46 | 0.46 | 0.43 |
| | (±0.032) | (±0.043) | (±0.071) |

(Figures in parenthesis indicates ±SE of mean values) N=6 (3 male+3 female rats/group)

Values in each of the first two columns above represent ratio of relative weight (calculated organ to body weight ratio) of spleen or thymus in drug treated animals v/s control animals. A ratio of 0.75 and above indicates minimal changes in the weight of the organs and the value of 1 suggests absence of adverse drug effect on spleen or thymus. The “Retics” column provides ratio of percentage reticulocytes in treated v/s percentage reticulocytes in control rats.

It should be noted here that none of the compounds of this invention nor pharmaceutically acceptable complexes or salts thereof have been found to have toxicity that would cause any problem.

Results:

Compound No.4 of the invention provided no significant changes of relative weight ratios of spleen- or thymus-weight to body weight with respect to untreated controls in contrast to compound No.30 of U.S. Pat. No. 5,668,286 or for Linezolid, which showed significant changes of relative weight ratios of spleen- or thymus-weight to body weight with respect to untreated controls. Furthermore, compound No.4 of the invention provided no significant change in ratio of percentage reticulocytes of treated animal versus control animal in comparison to values for compound No. 30 of U.S. Pat. No. 5,668,286 and for Linezolid.

The following examples are provided to further illustrate this invention but they should not be taken as limiting.

### EXAMPLE 1

### (S)-N-{3-[4-(4-cyanomethyl-piperidin-1-yl)-phenyl]-2-oxo-oxazolidin-5-ylmethyl}-formamide

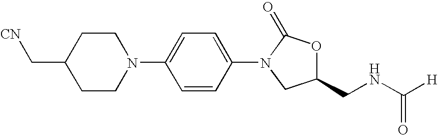

The suspension of (S)-N-{3-[4-(4-cyanomethylidene-piperidin-1-yl)-phenyl]-2-oxo-oxazolidin-5-ylmethyl}-formamide (1.12 mmol) and 10% palladium on carbon (0.1 g) in tetrahydrofuran was stirred under hydrogen atmosphere at room temperature for 8 hours. The suspension was filtered and the filtrate was concentrated to provide a title compound in 88% yield.

### EXAMPLE-2

### (S)-N-{3-[4-(4-cyanomethyl-piperidin-1-yl)-phenyl]-2-oxo-oxazolidin-5-ylmethyl}-acetamide

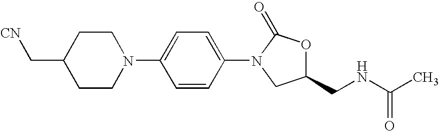

The title compound was prepared as per procedure described in Example-1 using (S)-N-{3-[4-(4-cyanomethylidene-piperidin-1-yl)-phenyl]-2-oxo-oxazolidin-5-ylmethyl}-acetamide in 90% yield.

Mp. 150-152° C. ¹H-NMR (CDCl₃, 200 MHz): δ 1.42-1.60 (m, 2H), 1.75-2.00 (m, 3H), 2.05 (s, 3H), 2.90 (d, 2H), 2.75 (m, 2H), 3.50-3.80 (m, 5H), 4.05 (m, 1H), 4.75 (m, 1H), 6.15 (t, 1H), 6.90 (dd, 2H), 7.40 (dd, 2H) MS (ES⁺): m/z=357.

### EXAMPLE-3

### (S)-N-{3-[4-(4-cyanomethyl-3-fluoropiperidin-1-yl)-phenyl]-2-oxo-oxazolidin-5-ylmethyl}-acetamide

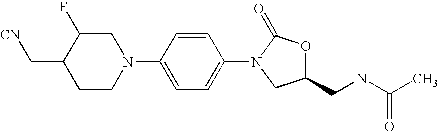

The title compound was prepared as per procedure described in Example-1 using (S)-N-{3-[4-(4-cyanomethylidene-3-fluoropiperidin-1-yl)-phenyl]-2-oxo-oxazolidin-5-ylmethyl}-acetamide in 91% yield.

MS (ES⁺): m/z=375.

### EXAMPLE-4

### (S)-N-{3-[4-(4-cyanomethyl-piperidin-1-yl)-3-fluoro-phenyl]-2-oxo-oxazolidin-5-ylmethyl}-acetamide

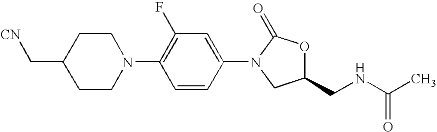

The compound was prepared as per procedure described in Example-1 by using (S)-N-{3-[4-(4-cyanomethylidene-piperidin-1-yl)-3-fluoro-phenyl]-2-oxo-oxazolidin-5-ylmethyl}-acetamide in 89% yield.

Mp. 220-222° C. ¹H-NMR (CDCl₃, 200 MHz): δ 1.49 (m, 2H), 1.75-2.00 (m, 3H), 2.05 (s, 3H), 2.30-2.41 (m, 2H), 2.60-2.80 (m, 2H), 3.38-3.50 (m, 2H), 3.60-3.81 (m, 3H), 3.95-4.10 (m, 1H), 4.70-4.85 (m, 1H), 6.41-6.59 (m, 1H), 6.90 (dd, 1H, J=9.2, 9.2 Hz), 7.10 (dd, 1H, J=2.2, 2.2 Hz), 7.41 (dd, 1H, J=2.2, 14.0 Hz) MS (ES⁺): m/z=375.

### EXAMPLE-5

### Preparation of Inclusion Complex of (S)-N-{3-[4-(4-cyanomethyl-piperidin-1-yl)-3-fluoro-phenyl]-2-oxo-oxazolidin-5-ylmethyl}-acetamide With 3-hydroxy-propyl-B-cyclodextrin (3-HP-B-CD) in 1:1.2 Molar Ratio

3-HP-B-CD (485 mg, 0.316 mmol) was dissolved in a 10 ml distilled water. To the clear solution, (S)-N-{3-[4-(4-cyanomethyl-piperidin-1-yl)-3-fluoro-phenyl]-2-oxo-oxazolidin-5-ylmethyl}-acetamide ( 100 mg, 0.26 mmol) was charged at 32° C. under stirring. The reaction mixture was stirred vigorously at a temperature 32° C. for 1 hours. The reaction mixture was evaporated to dryness under vacuum below 45° C. to provide a white solid in 575 mg quantity in quantitative yield.

Differential Scanning Colorimetry (DSC): The DSC spectrum of the inclusion complex did not show endotherm at 168° C., however the physical mixture in same molar ratio has shown the endotherm at 168.0° C. Powder X-ray diffractogram (XRPD): The XRPD of the inclusion complex showed amorphous nature of the complex where a hump was observed. However the powder X-ray difractogram of a physical mixture in same molar ratio showed peaks at 10.54, 17.60 and 21.32 (2θ values).

### EXAMPLE 5A

### Preparation of Inclusion Complex of (S)-N-{3-[4-(4-cyanomethyl-piperidin-1-yl)-3-fluoro-phenyl]-2-oxo-oxazolidin-5-ylmethyl}-acetamide with 3-hydroxy-propyl-B-cyclodextrin (3-HP-B-CD) in 1:2 Molar Ratio

3-HP-β-CD (12.30 gm, 8.03 mmol) was dissolved in a 150 ml distilled water. To the clear solution, (S)-N-{3-[4-(4-cyanomethyl-piperidin-1-yl)-3-fluoro-phenyl]-2-oxo-oxazolidin-5-ylmethyl}-acetamide ( 1.5 gm, 4.01 mmol) was charged at 30° C. under stirring. The resultant suspension was warmed to 48° C. for 2 hours to obtain a clear solution. The clear solution was cooled to a temperature at 25° C. and allowed to stand for 16 hours. The reaction mixture was filtered and filtrate was evaporated to dryness under vacuum below 45° C. temperature to provide a white solid in 13.0 gm quantity (90% yield).

Differential Scanning Colorimetry (DSC): The DSC spectrum of the inclusion complex did not show endotherm at 168° C., however the physical mixture in same molar ratio showed endotherm at 168.0° C. Powder X-ray diffractogram (XRPD): In the XRPD of the inclusion complex showed amorphous nature of the complex. However the powder X-ray difractogram of a physical mixture in same molar ratio showed peaks at 10.68, 17.78 and 21.44 (2θ values).

### EXAMPLE 6

### (S)-N-{3-[4-(4-cyanomethyl-piperidin-1-yl)-3-fluoro-phenyl]-2-oxo-oxazolidin-5-ylmethyl}-propionamide

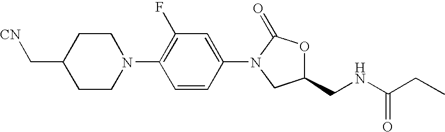

The compound was prepared as per procedure described in Example-1 by using (S)-N-{3-[4-(4-cyanomethylidene-piperidin-1-yl)-3-fluoro-phenyl]-2-oxo-oxazolidin-5-ylmethyl}-propionamide in 88% yield.

MS (ES⁺): m/z=389.

### EXAMPLE-7

### (S)-N-{3-[4-(4-cyanomethyl-piperidin-1-yl)-3-fluoro-phenyl]-2-oxo-oxazolidin-5-ylmethyl}-dichloroacetamide

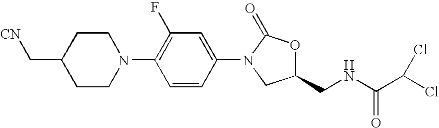

The compound was prepared as per procedure described in Example-1 by using (S)-N-{3-[4-(4-cyanomethylidene-piperidin-1-yl)-3-fluoro-phenyl]-2-oxo-oxazolidin-5-ylmethyl}-dichloroacetamide in 95% yield.

MS (ES⁺): m/z=443.

### EXAMPLE-8

### (S)-N-{3-[4-(4-cyanomethyl-piperidin-1-yl)-3-fluoro-phenyl]-2-oxo-oxazolidin-5-ylmethyl}-trifluoroacetamide

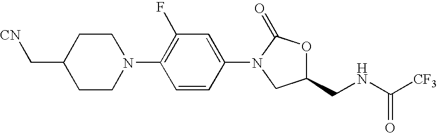

The compound was prepared as per procedure described in Example-1 in 71% yield.

Mp. 120-123° C. MS (ES⁺): m/z=429.

### EXAMPLE 9

### (S)-N-{3-[4-(4-cyanomethyl-piperidin-1-yl)-3-fluoro-phenyl]-2-oxo-oxazolidin-5-ylmethyl}-ethylcarbamate

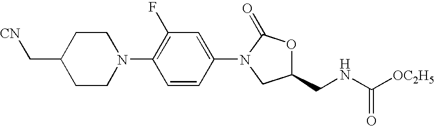

The compound was prepared as per procedure described in Example-1 in 67% yield.

Mp162-164° C. MS (ES⁺): m/z=375.

### EXAMPLE 10

### (S)-N-{3-[4-(4-cyanomethyl-piperidin-1-yl)-3-fluoro-phenyl]-2-oxo-oxazolidin-5-ylmethyl}-isobutylcarbamate

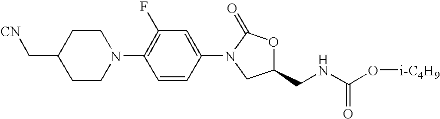

The compound was prepared as per procedure described in Example-1 in 79% yield.

Mp194-196° C. ¹H-NMR (CDCl₃, 200 MHz): δ 1.85 (dd, 6H), 1.50-1.70 (m, 2H), 1.80-2.0 (m, 4H), 2.40 (m, 2H), 2.60-2.80 (m, 2H), 3.40-3.60 (m, 4H), 3.70-3.90 (m, 3H), 4.05 (m, 1H), 4.70-4.85 (m, 1H), 6.90 (dd, 1H), 7.10 (dd, 1H), 7.40 (dd, 1H) MS (ES⁺): m/z=433.

### EXAMPLE 11

### (S)-N-{3-[4-(4-cyanomethyl-piperidin-1-yl)-3-fluoro-phenyl]-2-oxo-oxazolidin-5-ylmethyl}-t-butylcarbonylamide

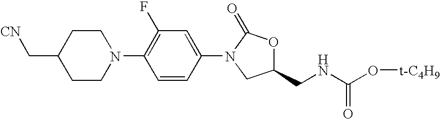

The compound was prepared as per procedure described in Example-1 in 71% yield.

Mp. 192-194° C. MS (ES⁺): m/z=433.

### EXAMPLE 12

### (S)-N-{3-[4-((4-cyanomethyl)-3-methyl-piperidin-1-yl)-3-fluoro-phenyl]-2-oxo-oxazolidin-5-ylmethyl}-acetamide

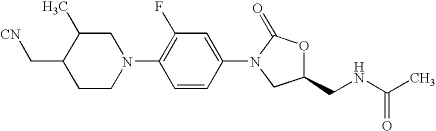

The compound was prepared as per procedure described in Example-1 in 75% yield.

M.P.=148-149° C. ¹H NMR (200 MHz, CDCl₃) □ ppm: 7.42 (dd, 1H, J=13.5, 1.8 Hz), 7.1 (dd, 1H, J=8.0, 1.8 Hz), 6.90 (t, 1H, J=8.0 Hz), 6.03 (t, 1H, D₂O exchangeable), 4.7-4.8 (m, 1H), 4.01 (t, 1H, J=8.0 Hz), 3.45-3.80 (m, 3H), 3.1-3.35 (m, 2H), 2.7-2.9 (m, 2H), 2.38 (d, 2H, J=7.0 Hz), 2.1-2.2 (m, 1H), 2.05 (s, 3H), 1.70-1.80 (m, 3H), 1.10 (d, 3H, J=7.0 Hz). MS (ES⁺): m/z=389.

### EXAMPLE 13

### (S)-N-{3-[4-((4-cyanomethyl)-3-fluoro-piperidin-1-yl)-3-fluoro-phenyl]-2-oxo-oxazolidin-5-ylmethyl}-isobutylcarbamate

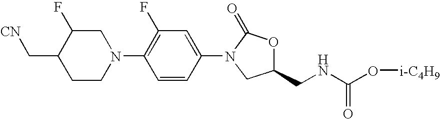

The compound was prepared as per procedure described in Example-1 in 81% yield.

Mp. 146-148° C. MS (ES⁺): m/z=451

### EXAMPLE 14

### (S)-N-{3-[4-(4-(1-cyanoethyl)-piperidin-1-yl)-3-fluoro-phenyl]-2-oxo-oxazolidin-5-ylmethyl}-acetamide

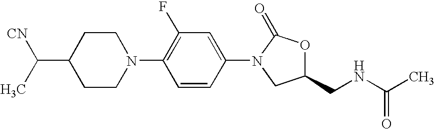

The compound was prepared as per procedure described in Example-1 in 87% yield.

M.P.=147-148° C. ¹H NMR (200 MHz, CDCl₃) □ ppm: 7.41 (dd, 1H, J=13.8, 1.8 Hz), 7.06 (dd, 1H, J=8/0. 1.8 Hz), 6.88 (t, 1H, J=8.0 Hz), 6.00 (t, 1H, D₂O exchangeable), 4.7-4.8 (m, 1H), 4.02 (t, 1H, J=7.0 Hz), 3.60-3.80 (m, 3H), 3.42 (bd, 2H), 2.45-2.70 (m, 3H), 2.03 (s, 3H), 1.6-1.8 (m, 5H), 1.38 (d, 3H, J=6.5 Hz). MS (ES⁺): m/z=389

### EXAMPLE 15

### (S)-N-{3-[4-(4-(1-cyanopropyl)-piperidin-1-yl)-3-fluoro-phenyl]-2-oxo-oxazolidin-5-ylmethyl}-acetamide

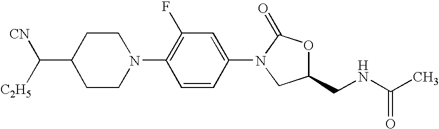

The compound was prepared as per procedure described in Example-1 in 88% yield.

Mp. 185-186° C. ¹H-NMR (CDCl₃, 200 MHz): δ 1.18 (t, 3H, J=4.8 Hz),1.60-1.89 (m, 4H), 2.05 (s, 3H), 2.30-2.50 (m, 1H), 2.60-2.80 (m, 2H), 3.39-3.60 (m, 2H), 3.60-3.82 (m, 3H), 3.90-4.10 (m, 1H), 4.70-4.85 (m, 1H), 5.95-6.19 (m, 1H), 6.90 (dd, 1H, J=9.2, 9.2 Hz), 7.05 (dd, 1H, J=2.2, 2.2 Hz), 7.41 (dd, 1H, J=2.2, 14.0 Hz). MS (ES⁺): m/z=403.

### EXAMPLE 16

### (S)-N-{3-[4-(4-(1-cyanobutyl)-piperidin-1-yl)-3-fluoro-phenyl]-2-oxo-oxazolidin-5-ylmethyl}-acetamide

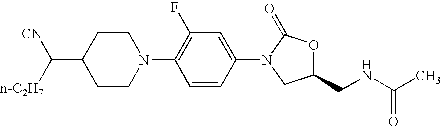

The compound was prepared as per procedure described in Example-1 in 82% yield.

Mp. 180-182° C. ¹H-NMR (CDCl₃, 200 MHz): δ 0.82-1.10 (m, 2H,), 1.20-1.40 (m, 2H), 1.55-1.80 (m, 7H), 2.05 (s, 3H), 2.40-2.80 (m, 3H), 3.40-3.55 (m, 2H), 3.60-3.80 (m, 3H), 3.95-4.10 (m, 1H), 4.70-4.85 (m, 1H), 6.15-6.25 (m, 1H), 6.90 (dd, 1H, J=9.2, 9.2 Hz), 7.05 (dd, 1H, J=2.2, 2.2 Hz), 7.44 (dd, 1H, J=2.2, 14.0 Hz). MS (ES⁺): m/z=417.

### EXAMPLE 17

### (S)-N-{3-[4-(4-(1-cyano-2-hydroxyethyl)-piperidin-1-yl)-3-fluoro-phenyl]-2-oxo-oxazolidin-5-ylmethyl}-acetamide

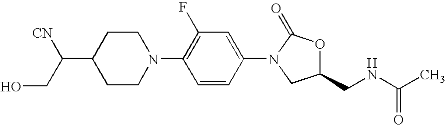

The compound was prepared as per procedure described in Example-1 in 62% yield.

M.P.=182-184° C. ¹H NMR (200 MHz, DMSO-d₆) □ ppm: 8.22 (1H, t, D₂O exchangeable), 7.43 (dd, 1H, J=13.8, 1.8Hz), 7.18 (dd, 1H, J=8.0, 1.8 Hz), 7.05 (t, 1H, J=8.0 Hz), 5.23 (bt, 1H, D₂O exchangeable), 4.30-4.80 (m, 1H), 4.05 (t, 1H, J=7.0 Hz), 3.60-3.75 (m, 3H), 3.25-3.40 (m, 4H), 2.82 (m, 1H), 2.60 (bt, 2H), 1.82 (s, 3H), 1.35-1.82 (m, 5H). MS (ES⁺): m/z=405

### EXAMPLE 18

### (S)-N-{3-[4-(4-(1-cyano-1-hydroxycarbonyl)-piperidin-1-yl)-3-fluoro-phenyl]-2-oxo-oxazolidin-5-ylmethyl}-acetamide

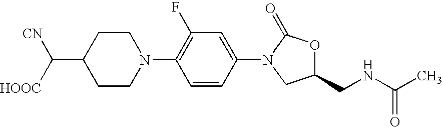

The compound was prepared as per procedure described in Example-1 in 62% yield.

M.P.=198-200° C. ¹H NMR (200 MHz, DMSO-d₆) □ ppm: 8.25 (1H, t, D₂O exchangeable), 7.43 (dd, 1H, J=13.3, 1.8Hz), 7.15 (dd, 1H, J=8.0, 1.8 Hz), 7.03 (t, 1H, J=8.0 Hz), 4.6-4.8 (M, 1H), 3.60-4.1 (m, 4H), 3.2-3.4 (m, 3H), 2.75 (bt, 2H), 1.9 (s, 3H), 1.45-1.8 (m, 5H). MS (ES⁺): m/z=419

### EXAMPLE 19

### (S)-N-{3-[4-(4-(1,1-dicyanomethyl)-piperidin-1-yl)-3-fluoro-phenyl]-2-oxo-oxazolidin-5-ylmethyl}-acetamide

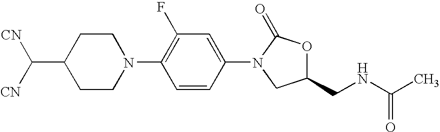

The compound was prepared as per procedure described in Example-1 in 60% yield.

Mp. 223-225° C. ¹H-NMR (CDCl₃, 200 MHz): δ 1.75-1.90 (m, 2H), 2.00 (s, 3H), 2.05-2.10 (m, 2H), 2.61-2.82 (m, 1H), 3.41-3.62 (m, 2H), 3.65-3.85 (m, 3H), 4.00-4.20 (m, 1H), 4.70-4.90 (m, 1H), 6.05 (t, 1H, J=5.9 Hz), ), 6.90 (dd, 1H, J=9.2, 9.2 Hz), 7.10 (dd, 1H, J=2.2, 2.2 Hz), 7.41 (dd, 1H, J=2.2, 14.0 Hz). MS (ES+): m/z=400.

1. An oxazolidinone antibiotic of Formula 1 an having in-vivo pharmacokinetic profile permitting of a once-a-day dosage regimen and having safety advantages in humans and animals, 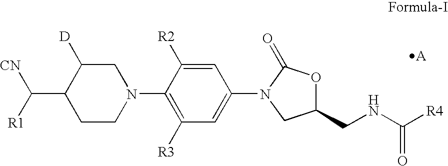
wherein,
R₁ is —H, C₁-C₈ alkyl, substituted alkyl, —COOH, or —CN;
R₂ and R₃ are the same or different and are H or fluorine;
R₄is H, C₁-C₈ alkyl, substituted C₁-C₈ alkyl, or C₁-C₈ alkoxy,;
D is H; C₁-C₈ alkyl, or fluorine and;
A is absent or is a complex forming agent, organic base, or amino acid.

2. A compound of the Formula 1 as claimed in claim 1 selected from the group consisting of:
(S)-N-{3-[4-(4-cyanomethyl-piperidin-1-yl)-phenyl]-2-oxo-oxazolidin-5-ylmethyl}-formamide;
(S)-N-{3-[4-(4-cyanomethyl-piperidin-1-yl)-phenyl]-2-oxo-oxazolidin-5-ylmethyl}-acetamide;
(S)-N-{3-[4-(4-cyanomethyl-3-fluoropiperidin-1-yl)-phenyl]-2-oxo-oxazolidin-5-ylmethyl}-acetamide;
(S)-N-{3-[4-(4-cyanomethyl-piperidin-1-yl)-3-fluoro-phenyl]-2-oxo-oxazolidin-5-ylmethyl}-acetamide;
(S)-N-{3-[4-(4-cyanomethyl-piperidin-1-yl)-3-fluoro-phenyl]-2-oxo-oxazolidin-5-ylmethyl}-acetamide, inclusion complex with 3-hydroxy-propyl-β-cyclodextrin;
(S)-N-{3-[4-(4-cyanomethyl-piperidin-1-yl)-3-fluoro-phenyl]-2-oxo-oxazolidin-5-ylmethyl}-propionamide;
(S)-N-{3-[4-(4-cyanomethyl-piperidin-1-yl)-3-fluoro-phenyl]-2-oxo-oxazolidin-5-ylmethyl}-dichloroacetamide;
(S)-N-{3-[4-(4-cyanomethyl-piperidin-1-yl)-3-fluoro-phenyl]-2-oxo-oxazolidin-5-ylmethyl}-trifluoroacetamide;
(S)-N-{3-[4-(4-cyanomethyl-piperidin-1-yl)-3-fluoro-phenyl]-2-oxo-oxazolidin-5-ylmethyl}-ethylcarbamate;
(S)-N-{3-[4-(4-cyanomethyl-piperidin-1-yl)-3-fluoro-phenyl]-2-oxo-oxazolidin-5-ylmethyl}-iso-butylcarbamate;
(S)-N-{3-[4-(4-cyanomethyl-piperidin-1-yl)-3-fluoro-phenyl]-2-oxo-oxazolidin-5-ylmethyl}-t-butylcarbamate;
(S)-N-{3-[4-((4-cyanomethyl)-3-methyl-piperidin-1-yl)-3-fluoro-phenyl]-2-oxo-oxazolidin-5-ylmethyl}-acetamide;
(S)-N-{3-[4-((4-cyanomethyl)-3-fluoro-piperidin-1-yl)-3-fluoro-phenyl]-2-oxo-oxazolidin-5-ylmethyl}-isobutylcarbamate;
(S)-N-{3-[4-(4-(1-cyanoethyl)-piperidin-1-yl)-3-fluoro-phenyl]-2-oxo-oxazolidin-5-ylmethyl}-acetamide;
(S)-N-{3-[4-(4-(1-cyanopropyl)-piperidin-1-yl)-3-fluoro-phenyl]-2-oxo-oxazolidin-5-ylmethyl}-acetamide;
(S)-N-{3-[4-(4-(1-cyanobutyl)-piperidin-1-yl)-3-fluoro-phenyl]-2-oxo-oxazolidin-5-ylmethyl}-acetamide;
(S)-N-{3-[4-(4-(1-cyano-2-hydroxyethyl)-piperidin-1-yl)-3-fluoro-phenyl]-2-oxo-oxazolidin-5-ylmethyl}-acetamide;
(S)-N-{3-[4-(4-(1-cyano-1-hydroxycarbonyl)-piperidin-1-yl)-3-fluoro-phenyl]-2-oxo-oxazolidin-5-ylmethyl}-acetamide;
(S)-N-{3-[4-(4-(1,1-dicyanomethyl)-piperidin-1-yl)-3-fluoro-phenyl]-2-oxo-oxazolidin-5-ylmethyl}-acetamide;

3. A process for preparation of a compound of Formula 1 as defined in claim 1 comprising:
(a) treating an appropriately substituted cyanoalkylidene piperidinophenyl oxazolidinone with hydrogen or hydrogen source in the presence of a catalyst and a solvent over a temperature range, to obtain a reaction mixture,
(b) filtering the reaction mixture to obtain a filtrate,
(c) concentrating the filtrate under vacuum to obtain a residue and
(d) purifying the residue to obtain a compound of Formula 1.

4. The process according to claim 3, wherein the hydrogen source is hydrogen gas at atmospheric pressure, ammonium formate or cyclohexene.

5. The process according to claim 3, wherein the hydrogen source is hydrogen gas at atmospheric pressure.

6. The process according to claim 3, wherein the catalyst is 5% palladium on carbon, 10% palladium on carbon or palladium hydroxide.

7. The process according to claim 3, wherein the catalyst is 10% palladium on carbon.

8. The process according to claim 3, wherein the solvent is selected from the group consisting of ethyl acetate, tetrahydrofuran, and methanol or mixtures thereof.

9. The process according to claim 3, wherein the solvent is selected from the group consisting of ethyl acetate and tetrahydrofuran.

10. The process according to claim 3, wherein the temperature range is between 20° C.-50° C.

11. A process for preparation of a complexed form of a compound according to claim 1 comprising:
(a) treating an appropriately substituted cyanoalkylidene piperidinophenyl oxazolidinone with hydrogen or hydrogen source in the presence of a catalyst and a solvent over a temperature range, to obtain a reaction mixture,
(b) filtering the reaction mixture to obtain a filtrate,
(c) concentrating the filtrate under vacuum to obtain a residue,
(d) purifying the residue to obtain a compound according to claim 1,
(e) treating the compound with a complex forming agent under stirring upto a temperature of 60° C. to obtain a solution;
(f) allowing the solution to stand at a temperature between 20-40° C. for 12-24 hrs,
(g) filtering the solution to obtain a filtrate,
(h) evaporating the filtrate under vacuum at a temperature below 60° C. to obtain a complexed form of the compound of claim 1.

12. The process according to claim 11, wherein the complex forming agent is selected from the group consisting of alpha cyclodextrin, beta cyclodextrin, substituted beta cyclodextrin and gamma cyclodextrin.

13. The process according to claim 11, wherein the complex forming agent is 3-hydroxypropyl beta cyclodextrin.

14. (cancelled)

15. (cancelled)

16. (cancelled)

17. A composition comprising an antibacterially effective amount of a compound according to claim 1 in admixture with one or more pharmaceutical carrier or excipient.

18. The composition of claim 17, adapted for oral, intravenous, topical, rectal, vaginal, or nasal administration.

19. The composition of claim 17, providing a pharmacokinetic profile consonant with a once-a-day dosage regimen in human or animal body.

20. The composition as claimed in claim 17, with a pharmacokinetic profile in a human or animal such that a concentration of the compound above its antibacterial minimum inhibitory concentration value is circulating in a human or animal blood stream for a period permissive of a once-a-day dosage regimen.

21. A composition as claimed in claim 17, with a pharmacokinetic T½ value in human or animal such that a concentration of the compound above its antibacterial minimum inhibitory concentration value is circulating in a human or animal blood stream for a period permissive of a once-a-day dosage regimen, when administered either orally or intravenously.

22. A composition as claimed in claim 17, which provides a blood concentration at 12 hrs of the active ingredient above the active ingredient's antibacterial minimum inhibitory concentration value, circulating in the blood of a human or animal blood stream for a period permissive of a once-a-day dosage regimen, when administered either orally or intravenously.

23. The composition as claimed in claim 17, with an overall pharmacokinetic profile in human or animal which is predictive of a once-a-day dosage regimen.

24. The composition of claim 17, which has safety advantage in human or animal such as that of a low or no potential to induce myelosuppression.

25. (cancelled)

26. (cancelled)

27. (cancelled)

28. (cancelled)

29. A method of combating a bacterial infection of the human or animal body which comprises administering to the body orally, parenterally, rectally, vaginally or nasally an effective amount of a compound as claimed in claim 1.

30. A method for treating a systemic or topical infection comprising administering an effective amount of a compound according to claim 1 to a patient in need thereof.

31. A method for treating a systemic or topical infection comprising administering an effective amount of a compound according to claim 1 to a patient in need thereof.

32. A method for preventing a systemic or topical infection comprising administering an effective amount of a compound according to claim claim 1 to a patient at risk for developing the infection.

33. A method for treating a systemic or topical infection comprising administering an effective amount of a compound according to claim claim 1 wherein the relative weight ratio of organ weight/body weight ratio in a treated animal is >0.75 as compared to a control animal.

34. A method for treating a systemic or topical infection comprising administering an effective amount of a compound according to claim 1 wherein the relative weights ratio of the spleen weight/body weight ratio in a treated animal is >0.75 as compared to a control animal.

35. A method for treating a systemic or topical infection comprising administering an effective amount of a compound according to claim 1 wherein the relative weights ratio of the thymus weight/body weight ratio in a treated animal is >0.75 as compared to a control animal.

36. A method for treating a systemic or topical infection comprising administering an effective amount of a compound according to claim 1 wherein the ratio of percentage reticulocyte counts in a treated animal is >0.75 as compared to percentage reticulocyte counts in a control animal.

37. A composition comprising an antibacterially effective amount of a compound according to claim 2 in admixture with one or more pharmaceutical carriers or excipients.

38. The composition of claim 37, adapted for oral, intravenous, topical, rectal, vaginal, or nasal administration.

39. A composition as claimed in claim 37, with a pharmacokinetic profile in a human or animal such that a concentration of the compound have its antibacterial minimum inhibitory concentration value is circulating in a human or animal blood stream for a period permissive of a once-a-day dosage regimen.

40. A composition as claimed in claim 37, with a pharmacokinetic T½ value in human or animal such that a concentration of the compound above its antibacterial minimum inhibitory concentration value is circulating in a human or animal blood stream for a period permissive of a once-a-day dosage regimen, when administered either orally or intravenously.

41. A composition as claimed in claim 37, which provides a blood concentration at 12 hrs of the active ingredient above the active ingredient's antibacterial minimum inhibitory concentration value, circulating in the blood of a human or animal blood stream for a period permissive of a once-a-day dosage regimen, when administered either orally or intravenously.

42. The composition of claim 37, which has safety advantage in human or animal such as that of a low or no potential to induce myelosuppression.

43. A method of combating bacterial infection of the human or animal body which comprises administering to the body orally, parenterally, rectally, vaginally or nasally an effective amount of a compound as claimed in claim 2.

44. A method of combating bacterial infection of the human or animal body which comprises administering to the body orally, parenterally, rectally, vaginally or nasally an amount of a composition according to claim 17 to combat the infection.

45. A method of combating bacterial infection of the human or animal body which comprises administering to the body orally, parenterally, rectally, vaginally or nasally an amount of a composition according to claim 37 to combat the infection.

46. A method for preventing a systemic or topical infection comprising administering an effective amount of a compound according to claim 2 to a patient at risk for developing the infection.

47. A method for treating a systemic or topical infection comprising administering an effective amount of a compound according to claim claim 2 wherein the relative weights ratio of organ weight/body weight ratio in a treated animal is >0.75 as compared to a control animal.

48. A method for treating a systemic or topical infection comprising administering an effective amount of a compound according to claim 2 wherein the relative weights ratio of the spleen weight/body weight ratio in a treated animal is >0.75 as compared to a control animal.

49. A method for treating a systemic or topical infection comprising administering an effective amount of a compound according to claim 2 wherein the relative weights ratio of the thymus weight/body weight ratio in a treated animal is >0.75 as compared to a control animal.

50. A method for treating a systemic or topical infection comprising administering an effective amount of a compound according to claim 2 wherein the ratio of percentage reticulocyte counts in a treated animal is >0.75 as compared to percentage reticulocyte counts in a control animal.
